(19) United States
(12) Patent Application Publication
(10) Pub. No.: US 2007/0100366 A1
(43) Pub. Date: May 3, 2007

(54) Minimally invasive tissue expander systems and methods

(76) Inventors: Sara Dziedzic, Braintree, MA (US); Connie Marchek, Foxboro, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/260,972

(22) Filed: Oct. 28, 2005

(51) Int. Cl. A61M 29/00

(52) U.S. Cl. 606/191

(57) ABSTRACT

A tissue expander system for expanding tissue, skin and muscle along a pathway to a surgical site through a minimally invasive incision includes a first paddle, a second paddle and an outer sleeve. The first and second paddles connect to form an assembly for the outer sleeve to advance over. An access device can be inserted over the outer sleeve to form the pathway to the surgical site after the tissue expander system is removed.

### BACKGROUND

In minimally invasive surgical procedures providing access to the surgical site is a balancing act between minimizing the size of the incision and providing enough room for the surgeon to manipulate the instruments to perform the surgery. Access devices, including expandable tubular retractors and ports are used to retract the skin, muscles and tissue from the surface of the skin to the surgical site providing an unobstructed pathway for the surgeon to work. Typically, a sequential dilation technique is used to insert an access device. To minimize the damage to the tissue and muscles in creating a pathway, a small incision is made in the skin and a guide wire is inserted. Next a small diameter tubular member is advanced over the guide wire until it reaches the desired surgical site. As the tube is advanced it pushes the skin and tissue out of the way creating the surgical path. A second tubular member having a slightly larger diameter is then advanced over the first tubular member creating a wider path. These steps are repeated using tubular members of increasing diameter until the desired size of the path is created. Finally, an access device is advanced over the largest tubular member and the tubular members are removed leaving the access device in place. Using this technique minimizes trauma to the tissue as the tissue is expanded or stretched rather than cut as in an open procedure.

Some drawbacks with the dilator system include the number of steps it takes to create the desired size for the access device, and difficulty in inserting and removing the tubular members.

### SUMMARY

Disclosed herein are tissue expander systems and methods of use. In one embodiment a tissue expander system may include a first paddle having a connecting feature and a second paddle having a connecting feature adapted to connect to the connecting feature of the first paddle forming an assembly of the first and second paddles. The system may further include an outer sleeve adapted to slide over the first and second paddles. The outer sleeve may also have slots to receive the first and second paddles. The system may also include a handle for insertion and/or removal of the paddles.

In an alternate embodiment the tissue expander system may include first and second paddles having mating channels for connecting the paddles. The system may also include an outer sleeve with slots for receiving the paddles. In yet another embodiment, the tissue expander system may include first and second paddles and an access device for creating a pathway to the surgical site.

The surgeon when using the tissue expander system inserts a first paddle through an incision into proximity with the vertebral body; inserts a second paddle through the incision; connects the second paddle to the first paddle; inserts an outer sleeve over the first and second paddles into proximity with the vertebral body; and places an access device over the outer sleeve.

In an alternate use, the surgeon may insert a paddle through an incision to a point proximate the vertebral body; rotate the paddle within the incision; and insert an access device over the paddle to create a pathway to the vertebral body. The surgeon may choose to add an outer sleeve over the paddle prior to placing the access device.

In yet another alternate method of creating a minimally invasive pathway to a vertebral body, the surgeon may insert a paddle through an incision to a point proximate the vertebral body; insert a second paddle within the incision in connection with the first paddle; and insert an access device over the first and second paddles to create a pathway to the vertebral body.

### BRIEF DESCRIPTION OF THE FIGURES

These and other features and advantages of the tissue expander system and methods disclosed herein will be more fully understood by reference to the following detailed description in conjunction with the attached drawings in which like reference numerals refer to like elements through the different views. The drawings illustrate principles of the tissue expander system and methods disclosed herein and, although not to scale, show relative dimensions.

FIG. 1A illustrates a perspective view of the first paddle of the tissue expander system.

FIG. 1B illustrates a side view of the first paddle shown in FIG. 1A.

FIG. 1C illustrates an end view of the first paddle shown in FIG. 1A.

FIG. 2A illustrates a perspective view of the second paddle of the tissue expander system.

FIG. 2B illustrates a side view of the second paddle shown in FIG. 2A.

FIG. 2C illustrates an end view of the second paddle shown in FIG. 2A.

FIG. 3A illustrates a perspective view of the outer sleeve of the tissue expander system.

FIG. 3B illustrates a side view of the outer sleeve shown in FIG. 3A.

FIG. 3C illustrates an end view of the outer sleeve shown in FIG. 3A.

FIG. 4A illustrates a perspective view of the paddle insertion handle for the tissue expander system.

FIG. 4B illustrates a side view of the paddle insertion handle shown in FIG. 4A.

FIG. 5 illustrates a view of the assembly of the first and second paddles shown in FIGS. 1 and 2.

FIG. 6A illustrates a perspective view of the assembly of the second paddle, first paddle and the outer sleeve.

FIG. 6B illustrates a perspective view of the assembly shown in FIG. 6A.

FIG. 6C illustrates an end view of the assembly shown in FIG. 6A.

FIG. 6D illustrates the assembly of the first and second paddle with an access device.

FIG. 7 illustrates a cross-section of an assembly of a first paddle, a second paddle and an outer sleeve having docking features.

FIGS. 8A-D illustrate another embodiment of a tissue expander system.

FIGS. 9A-C illustrate another embodiment of an elliptically shaped tissue expander system.

FIGS. 10A-D illustrate another embodiment of a tissue expander system having only one paddle.

### DETAIL DESCRIPTION OF EXEMPLARY EMBODIMENTS

Certain exemplary embodiments will now be described to provide an overall understanding of the principles of the structure, function, manufacture, and use of the tissue expander system and methods disclosed herein. One or more examples of these embodiments are illustrated in the accompanying drawings. Those of ordinary skill in the art will understand that the tissue expander system and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The articles “a” and “an” are used herein to refer to one or to more than one (i.e. to at least one) of the grammatical object of the article. By way of example, “an element” means one element or more than one element.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

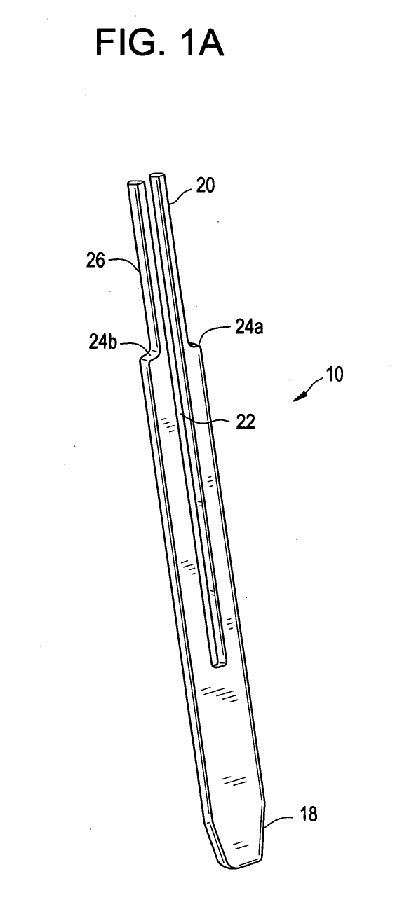

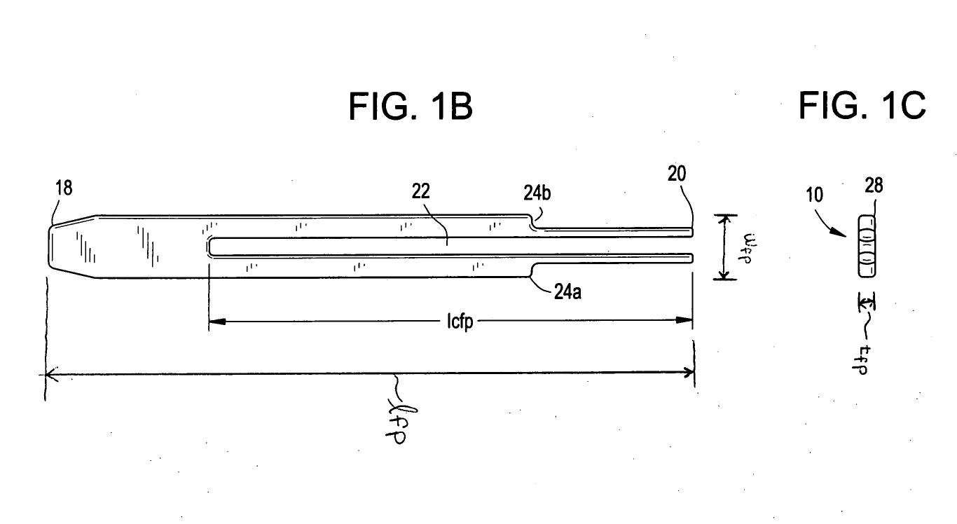

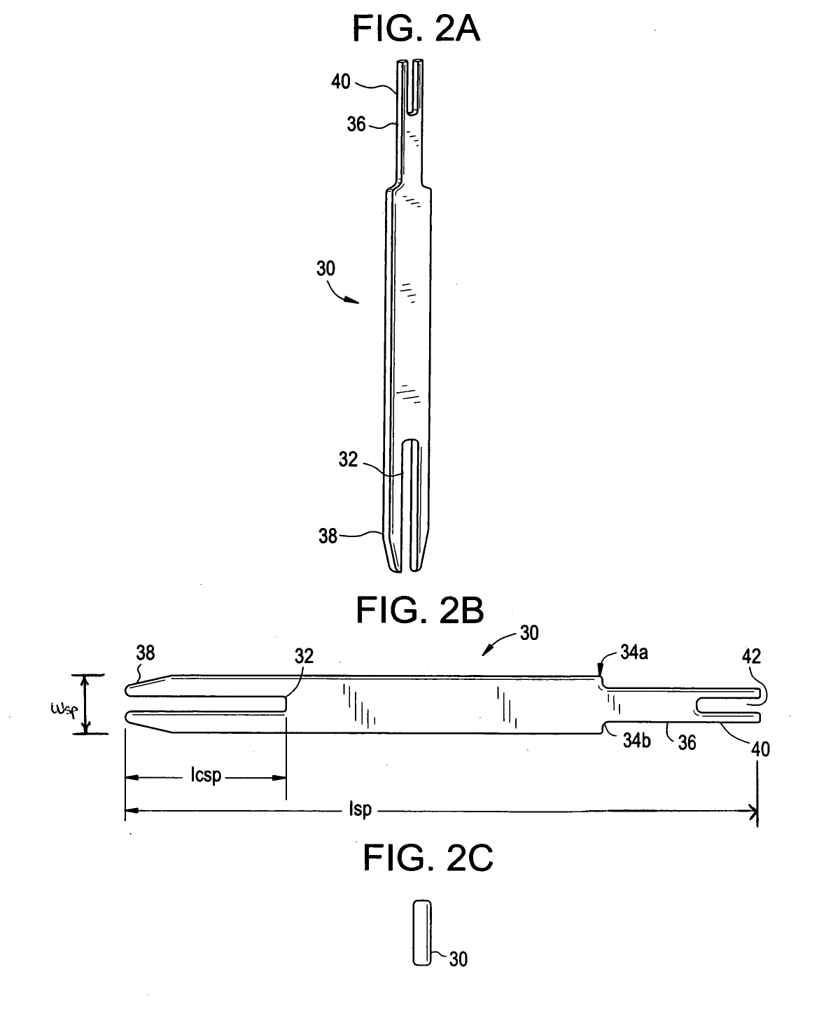

FIGS. 1-10 illustrate various components of an exemplary embodiment of a tissue expander system. The system may be used for numerous surgical procedures but will be described below in relation to spinal surgery. One skilled in the art will understand that the system can also be used for other surgical procedures including orthopedic. The system may be used with any number of surgical approaches used in spinal surgery including anterior, posterior, anterior/lateral, lateral, and posterior/lateral.

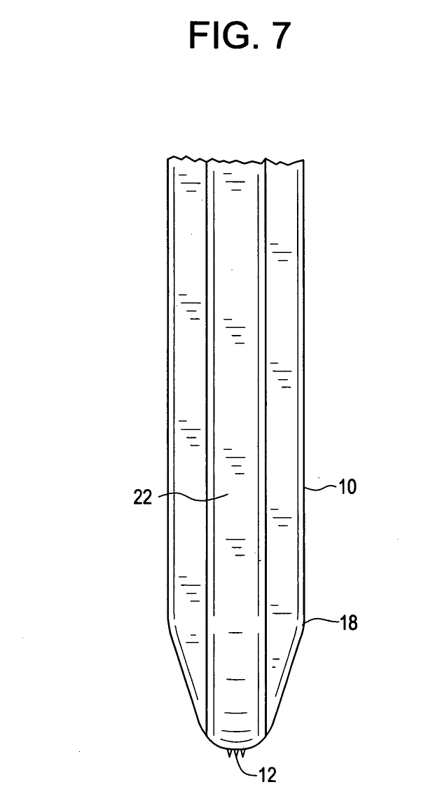

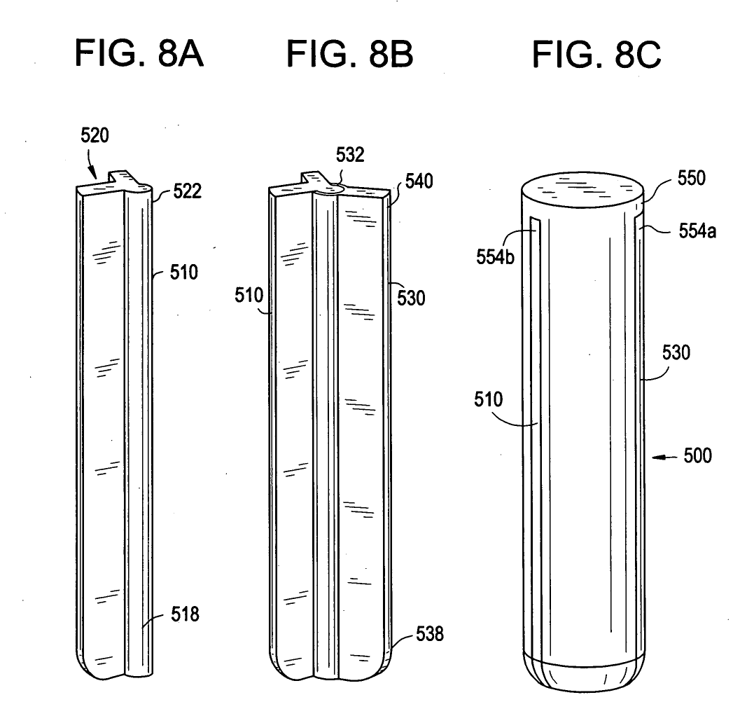

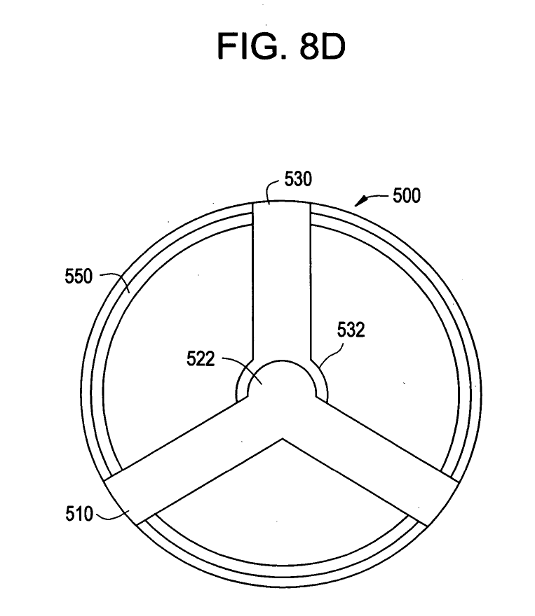

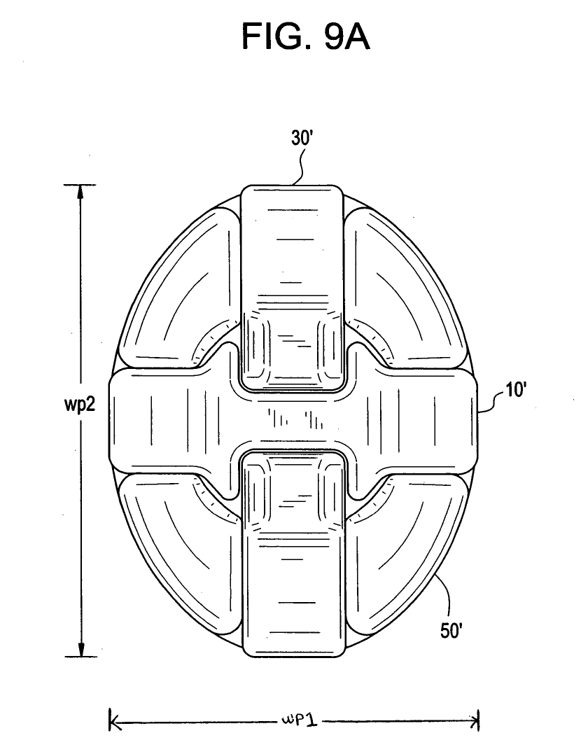

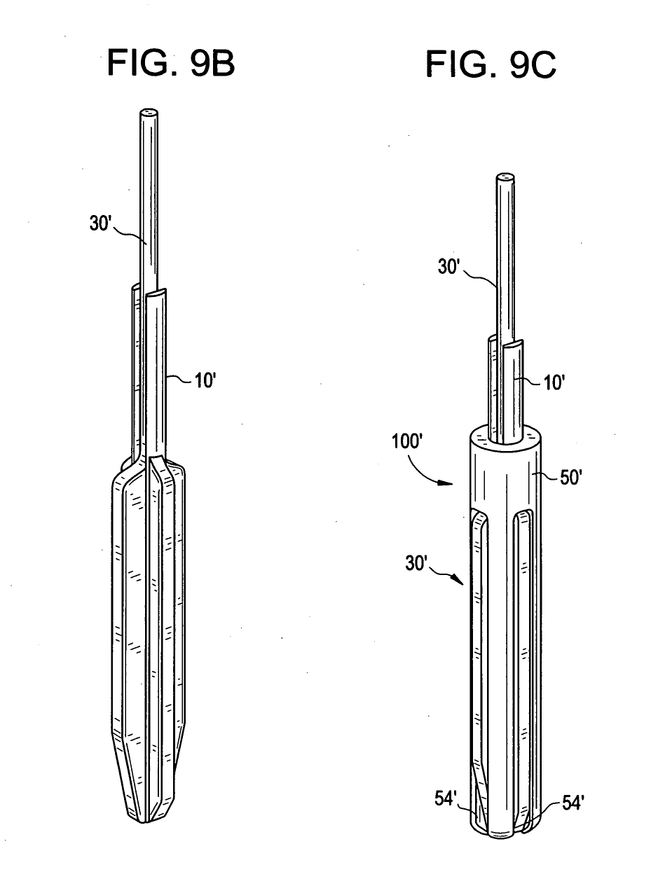

The exemplary tissue expander assembly 100 shown in FIG. 6A includes a first paddle 10, a second paddle 30 and an outer sleeve 50. The exemplary assembly 100 may be employed to expand skin, tissue, and muscle through a minimally invasive incision to create a pathway to a surgical site, i.e., a vertebral body. For example, the first paddle 10 is inserted through a skin incision and advanced proximate to the vertebral body. The first paddle 10 maybe rotated 90 degrees to expand or stretch the surrounding tissue, muscle and skin. The second paddle 30 is then inserted in connection with the first paddle 10 to form an assembly within the incision as shown in FIG. 5. The orientation of the paddles with respect to each other may be varied such that the angle between the first paddle 10 and the second paddle 30 may be acute (<90 degrees), obtuse (>90 degrees) or right (=to 90 degrees). Depending on the shape of the first and second paddles other various-shaped assemblies could also be constructed. The outer sleeve 50 is advanced over the assembly forming a circular or ellipsoidal shaped assembly and creating a pathway to the surgical site. An access device 200, such as a port or expandable retractor, may be advanced over the assembly to maintain the pathway during the surgical procedure. The tissue expander assembly 100 may be removed in one step, i.e., the first paddle 10, the second paddle 30 and the outer sleeve 50 may be removed simultaneously. The assembly 100, when employed in the exemplary manner, thus may be used to expand or dilate tissue, muscle or skin to create a pathway to the surgical site.

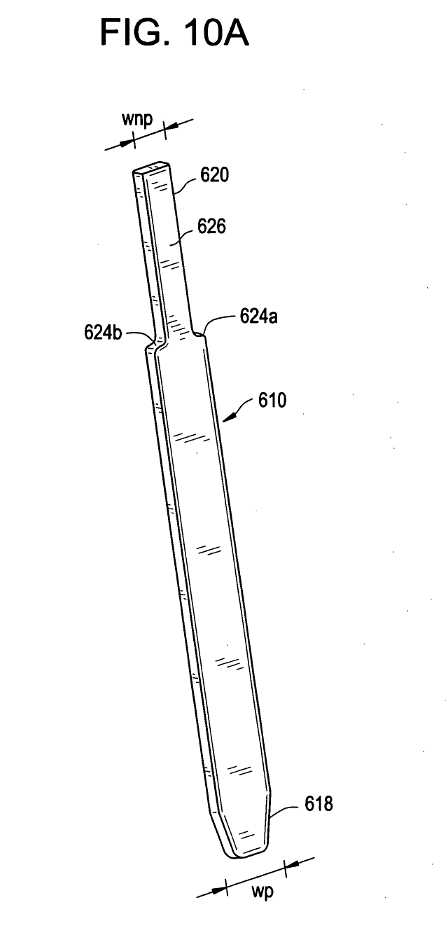

The first paddle 10 of the exemplary assembly 100 illustrated in FIGS. 1A and 1B extends from a distal end 18 to a proximal end 20, along a longitudinal axis and has a connecting feature 22 adapted to connect the first paddle 10 to the second paddle 30. The connecting feature 22 may be in the form of a tongue and groove, complementary mating shapes, a channel or other structures suitable for connecting the first paddle 10 to the second paddle 30. In the exemplary embodiment, the first paddle 10 is generally rectangular in shape having radiused edges 28 as seen in FIG. 1C. One skilled in the art will appreciate that the first paddle 10 may have other cross sectional shapes including, for example, v-shaped or elliptical. The distal end 18 of the first paddle 10 may be tapered for ease of insertion. The distal end 18 may also have features 12 to aid in docking to bone at the surgical site such as serrated edges, teeth, or roughened surface area. An example of a distal end 18 of a first paddle 10 having teeth 12 is shown in FIG. 7.

In the exemplary embodiment illustrated in FIGS. 1A and 1B, the connecting feature 22 of the first paddle 10 is a channel extending distally from the proximal end 20. The first paddle 10 has a length l_{cfp}, width w_{fp} and thickness t_{fp}. The length l_{fp} is such that the proximal end 20 of the first paddle 10 will extend above the skin incision. The channel 22 may extend over a portion of the first paddle 10 to a length l_{cfp} such that the percentage of l_{cfp} to l_{fp} is between 5% and 95%. The width w_{fp} of the first paddle 10 may be substantially constant between the proximal 20 and distal end 18 portions and may narrow near the proximal end 20 creating shoulders 24a,b. The narrow portion 26 of the first paddle 10 extending past the shoulders 24a,b may be used as a handle or to support a handle 80 for insertion or removal. The channel 22 is sized to receive a connecting feature 32 of the second paddle 30. The width of the channel 22 may be between 5% and 95% of the width of the first paddle 10. The width of the first paddle 10 may correlate to the desired size of the access device 200 to be used for the surgery.

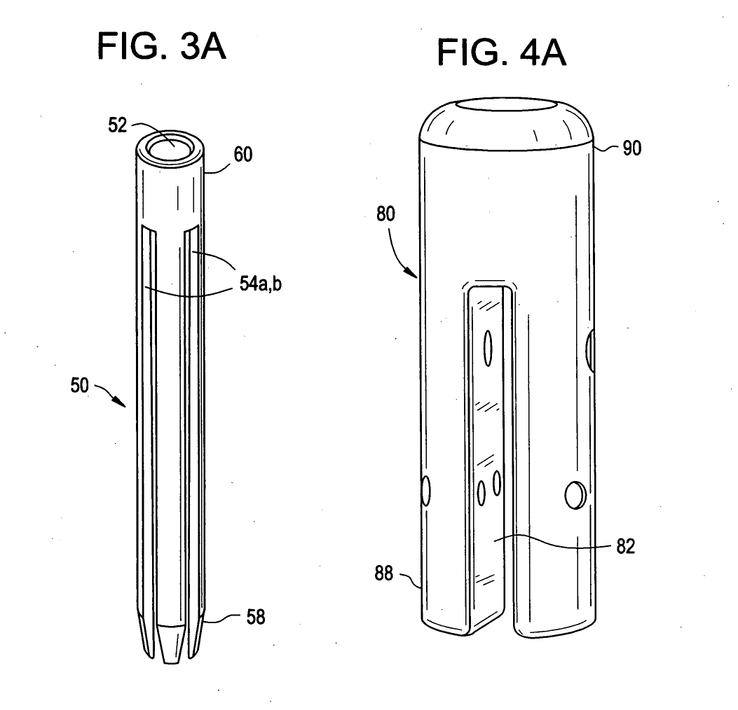

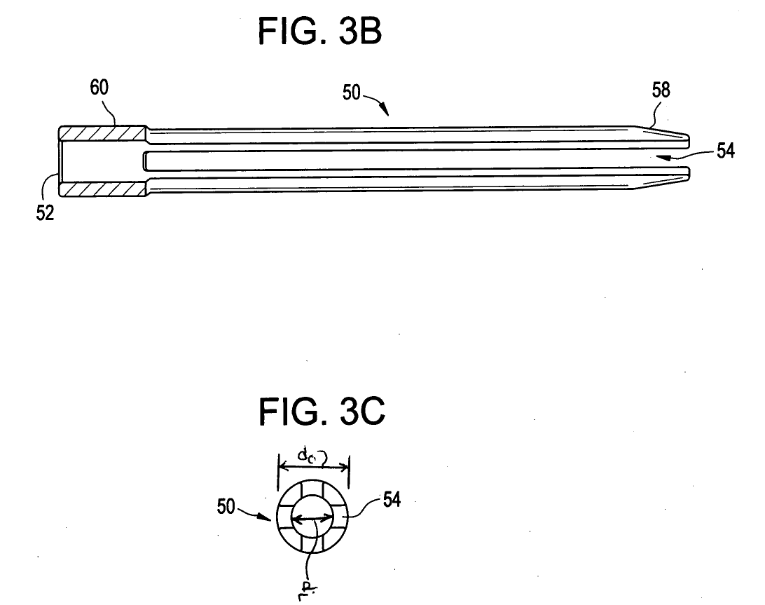

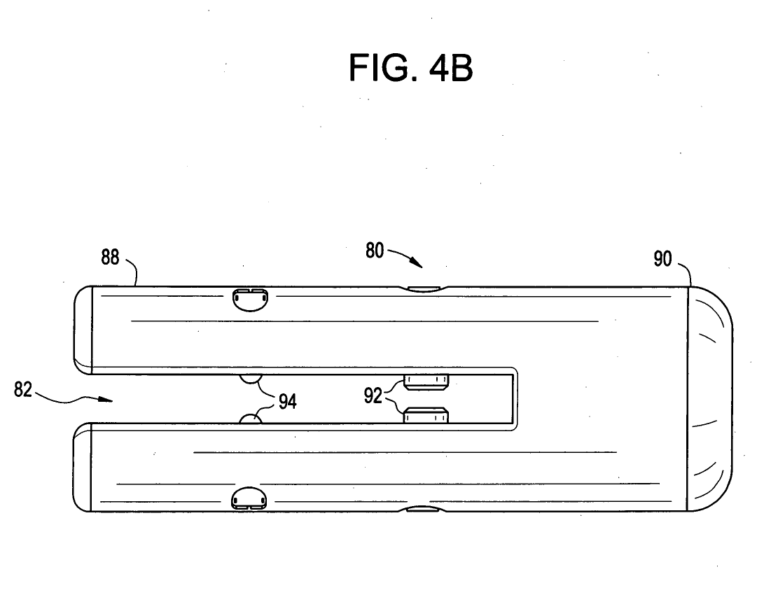

The exemplary embodiment of the second paddle 30 illustrated in FIGS. 2A-C has a distal end 38, a proximal end 40 and a connecting feature 32 adapted to connect with the connecting feature 22 of the first paddle 10 to connect the two paddles. The connecting feature 32 is exemplified as a channel extending along a portion of the second paddle 30 from the distal end 38. The distal end 38 of the second paddle 30 may be tapered for ease of insertion. The distal end 38 may also have the same feature 12 for docking to the bone at the surgical site as the first paddle 10. The second paddle 30 has an overall length lₛₚ from the proximal end 40 to the distal end 38. The channel 32 has a length l_{csp} extending from the distal end 38 between 5% and 95% of lₛₚ the length of the second paddle 30. The second paddle 30 has a width wₛₚ, which may be equal to the width of the first paddle 10. The width wₛₚ of the second paddle 30 may be substantially constant between the proximal end 40 and distal end 38 and may narrow near the proximal end 40 creating shoulders 34a,b. The narrow portion 36 of the second paddle 30 extending past the shoulders 34a,b may be used as a handle or to support a handle 80 for insertion or removal. The narrow portion 36 may be a solid shaft extending proximally from the shoulders or may have a channel 42 extending distally from the proximal end 40 for receiving a handle as illustrated in FIG. 2A.

The thickness of the second paddle 30 may be sized to be received within the channel 22 of the first paddle 10 creating a friction fit between the second paddle 30 and the first paddle 10. Alternately, the connecting feature 22 of the first paddle 10 and the connecting feature of the second paddle 30 may have interlocking features such as tongue and groove, ball plunger and detent, or pin and slot. The connecting features 22, 32 may have different complementary configurations to engage each other. The connecting features 22,32 may also be angled with respect to each other to alter the paddle assembly shape such that the angles between the first paddle 10 and the second paddle 30 may be acute (<90 degrees), obtuse (>90 degrees) or right (=90 degrees).

The first paddle 10 is inserted through a minimally invasive incision such that the width of the paddle w_{fp} is parallel to the longitudinal axis of the incision. Once the first paddle 10 is inserted to the desired depth it may be rotated approximately 90 degrees to further separate the tissue and muscle fibers leading to the surgical site proximate a vertebral body. The second paddle 30 is placed through the incision such that the connecting feature 32 of the second paddle 30 is aligned with the connecting feature 22 of the first paddle 10. The second paddle 30 is advanced along the channel 22 until the distal portion 38 of the second paddle 30 reaches the surgical site proximate the vertebral body. As the second paddle 30 is inserted it expands the tissue and muscles within the pathway to the surgical site proximate the vertebral body. As shown in FIG. 5 the connected first paddle 10 and the second paddle 30 are at right angles to each other forming a +-shape assembly.

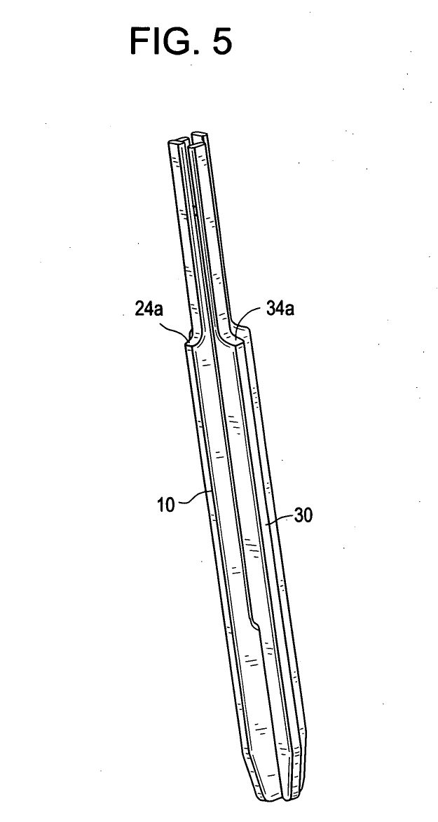

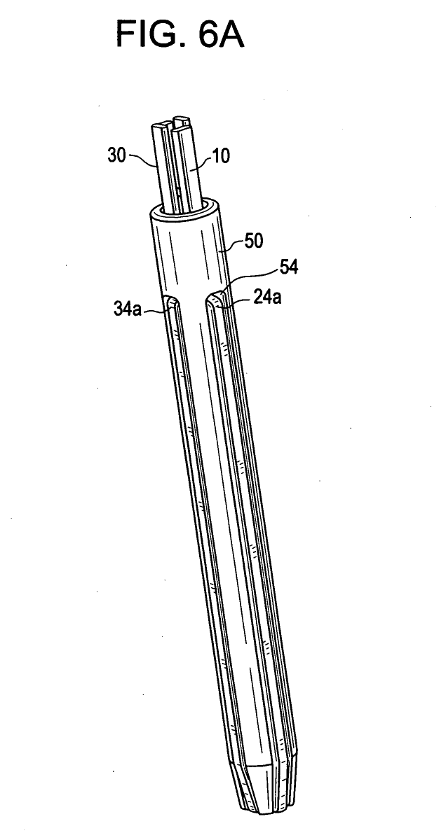

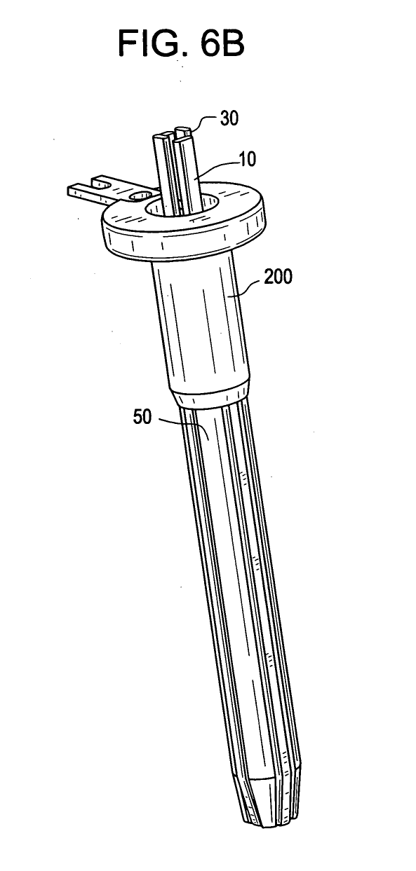

The outer sleeve 50 of the tissue expander assembly 100 shown in FIGS. 3A and 3B has a generally tubular shape with a central lumen 52 extending from a distal 58 end to a proximal 60 end. The outer sleeve 50 is sized to slide over the assembly of the first 10 and second 30 paddles. Extending proximally from the distal end 58 along the longitudinal axis of the outer sleeve 50 are slots 54a,b,c,d. As illustrated in FIG. 6B, each slot 54 is sized to receive a portion of the second paddle 30 or first paddle 10. The length of each slot 54 is adapted to accommodate the shoulders 24a,b and 34a,b formed on the first 10 and second 30 paddles. The distal end 58 of the outer sleeve 50 may be tapered to ease insertion. The length of the outer sleeve 50 is less than the overall length of the first 10 and second 30 paddles, such that the proximal ends 20, 40 of the first paddle 10 and second paddle 30 extend through the lumen of the outer sleeve 50. The outer sleeve 50 may have a generally circular or ellipsoidal shape depending on the shape of the access device 200 to be used. As shown in FIG. 3C, the inner diameter dᵢ of the lumen 52 of the outer sleeve 50 is smaller than the width w_{fp} of the first paddle 10 and the width wₛₚ of the second paddle 30 such that the narrow portion 26, 36 of the first 10 and second 30 paddles fits within the lumen 52 of the outer sleeve 50. The outer diameter d₀ of the outer sleeve 50 is the same as or greater than the width w_{fp} of the first paddle 10 and the width wₛₚ of the second paddle 30. The slots 54a,b,c,d of the outer sleeve 50 are sized to accommodate the width of the first and second paddles such that when the outer sleeve 50 is placed over the paddles the circumference of the outer sleeve is completed by the assembly.

The outer sleeve 50 is advanced through the incision over the first 10 and second 30 paddles. The slots 54a,b,c,d slide over the widest portion of the first 10 and second 30 paddles until the shoulders 24a,b and 34 a,b abut the proximal end of the slots 54, as shown in FIG. 6A. The first 10 and second 30 paddles fill the slots 54a,b,c,d of the outer sleeve 50 creating a tissue expander assembly 100 that retracts tissue and muscles along the pathway to the surgical site. FIG. 6C illustrates the completed circumference of the outer sleeve 50 with an end view from the bottom of the assembly 100. The access device 200, shown in FIG. 6B may be placed over the tissue expander assembly 100 to create the pathway to the surgical site. The tissue expander assembly 100 may be removed in one step by grabbing the first paddle 10 by the proximal end 20, leaving the access device 200 in place. Alternately, as shown in FIG. 6D, the outer sleeve 50 may not be used and the access device 200 may be placed directly over the assembly of the first paddle 10 and the second paddle 30.

In an alternate method, a guide wire can be inserted at the desired surgical site location. The first paddle may be cannulated (not shown) and inserted over the guide wire. After the first paddle is inserted, the guide wire is removed and the second paddle inserted as described in the method above.

The first paddle 10, the second paddle 30, and the outer sleeve 50 may be provided in varying sizes (diameters and lengths) correlating to the size of the access port 200 to be used. The paddles may range in diameter size from 10 mm to 35 mm, preferably from 13 mm to 26 mm. The second paddle 30 may also have depth markings for measuring the depth at the skin incision for assistance in determining the length of the access device 200 to be used. The paddles 10, 30 and outer sleeve 50 may be manufactured from any biocompatible material such as metal, plastic, or composite and may be radiopaque or radiolucent. If radiolucent, the paddles and or outer sleeve may also include depth markers made from radiopaque rings for intra-operative depth measurements under fluoroscopy. Alternately, the distal ends of the paddles and sleeve may be radiopaque to aid the surgeon under fluoroscopy.

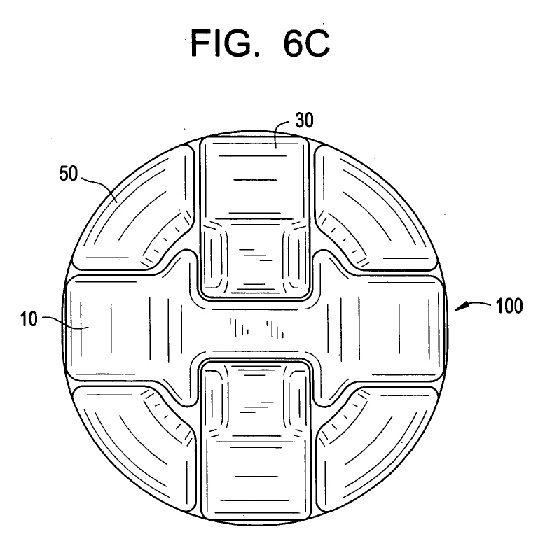

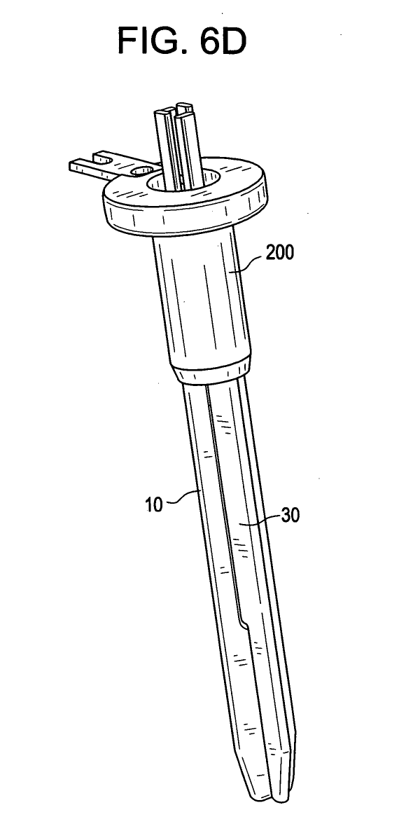

An insertion handle 80 shown in FIG. 4A may be used to insert the first 10 and second 30 paddles. The handle 80 has a generally cylindrical configuration having a distal portion 88 and a proximal portion 90. A first channel 82 extends through the handle 80 from the distal portion 88 towards the proximal portion 90. Within the channel 82 are connecting features 94 to connect the handle 80 to the first paddle 10. The connecting features 94 may include friction fit, ball plungers, spring loaded pins, grooves and detents. Alignment features 92 may be placed within the channel 82 to aid with alignment of the proximal end 20 of the paddle 10 during connection to the handle 80. The handle 80 may be connected to the first paddle 10 prior to insertion within the skin incision. After the first paddle 10 is inserted the handle 80 may then be removed from first paddle 10 and connected to the second paddle 30 for insertion. In an alternate embodiment the handle 80 may have multiple intersecting channels for connecting to both the first paddle 10 and the second paddle 30 simultaneously. The multiple channels allow the handle 80 to connect to both first 10 and second 30 paddles at the same time providing for easier insertion of the second paddle and/or removal of the entire assembly 100 in one step.

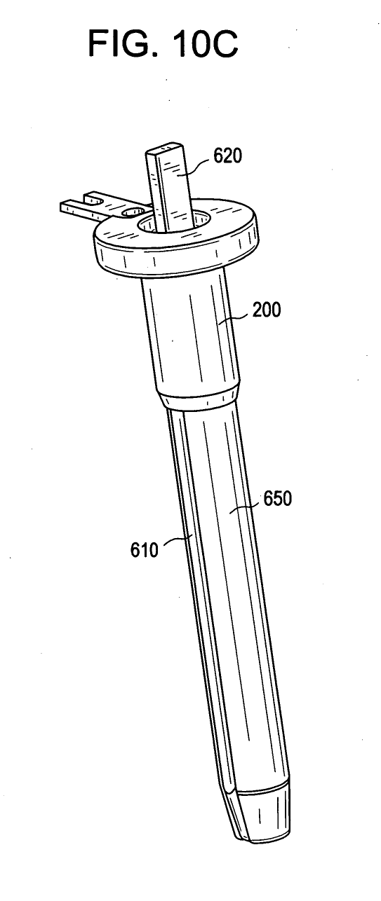

An alternate embodiment of a tissue expander system 100′ may have a generally elliptical shape to accommodate an elliptical access device such as a port or expandable retractor as illustrated in FIGS. 9A-C. The elliptical shaped tissue expander system 100′ includes a first paddle 10′, a second paddle 30′ and an outer sleeve 50′ similar to the system 100 described above. In this embodiment the second paddle 30′ may have a width wₚ₂ greater than the width of the first paddle wₚ₁ 10′ to create a t-shaped assembly. Alternately, the first paddle 10′ may have a width wₚ₁ greater than the width of the second paddle wₚ₂ 30′. The outer sleeve 50′ has a generally elliptical shape. Slots 54′ are configured to slide over the paddle assembly. The paddles are inserted as above and the outer sleeve 50′ is advanced over the paddle assembly. An elliptical shaped access device, such as a port or expandable retractor, not shown, is placed over the system 100′.

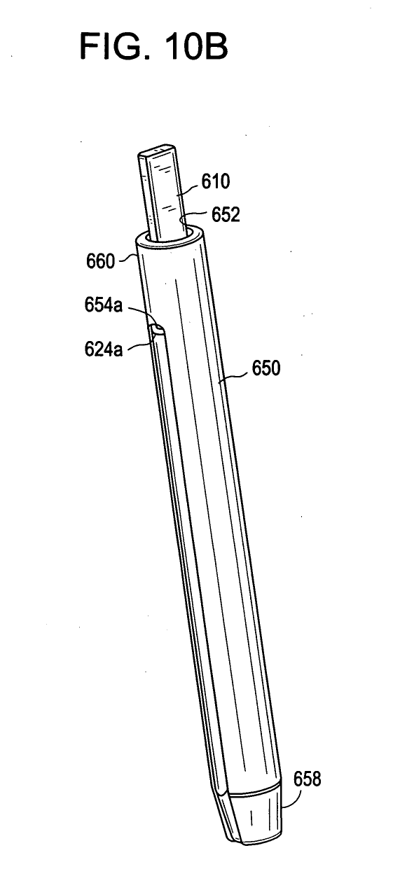

Another embodiment of a tissue expander system is illustrated in FIGS. 8A-D. The system 500 has components similar to the first system 100 including a first paddle 510, a second paddle 530, and an outer sleeve 550. In this embodiment the first paddle 510 and second paddle 530 have different shapes and connecting features from the first embodiment while the outer sleeve 550 remains substantially the same. The first paddle 510 has a distal end 518, a proximal end 520, and a connecting feature 522 adapted to connect the first paddle 510 to the second paddle 530. The first paddle 510 extends along a longitudinal axis between the proximal end 520 and the distal end 518. In the exemplary embodiment, the first paddle 510 is generally v-shaped and has radiused edges as seen in FIG. 8A. The distal end 518 of the first paddle 510 may be tapered for ease of insertion. The distal end 518 may also have features to aid in docking to bone at the surgical site such as serrated edges, teeth, or roughened surface area.

The first paddle 510 has a length l_{fp}, such that the proximal end 520 of the first paddle 510 will extend above the skin incision. The connecting feature 522 may extend over the entire length of the paddle or only a portion of the first paddle 510. A shaft or handle (not shown) may extend from the proximal end 520 of the first paddle 510 for inserting and removing the paddle. The connecting feature 522 is adapted to connect with a complementary shaped connecting feature on the second paddle 530. As shown in FIG. 8A the connecting feature 522 is in the form of a rounded projection.

The second paddle 530 illustrated in FIG. 8B has a distal end 538, a proximal end 540 and a complementary connecting feature 532, in this embodiment shown as a rounded channel. The second paddle 530 has a generally rectangular shape and has radiused edges. The distal end 538 of the second paddle 530 may be tapered for ease of insertion. The distal end 538 may also have the same feature for docking to the bone at the surgical site as the first paddle 510. The second paddle 530 has an overall length lₛₚ from the proximal end 540 to the distal end 538. The width of the second paddle 530 may correlate to the radius of the desired size of the access device to be used for the surgery. A shaft or handle (not shown) may extend from the proximal end of the second paddle 530 for use during insertion or removal of the paddle. The connecting feature 532 of the second paddle 530 as shown in FIG. 8B is a rounded channel complementary to the shape of the rounded projection 522 on the first paddle 510. Other complementary connecting features may include a dovetail, ball plunger, pin and slot, or detent. The connecting features 522,532 may also be angled with respect to each other to alter the paddle assembly shape, for example the paddles may be angled at acute, obtuse or right angles to each other.

The first paddle 510 is inserted through a minimally invasive incision. Once the first paddle 510 is inserted to the desired depth it may be rotated to further separate the tissue and muscle fibers leading to the surgical site. The second paddle 530 is inserted into the incision such that the connecting feature 532 connects with the connecting feature 522 of the first paddle 510. When the first paddle 510 has a v-shape and the second paddle 530 has a rectangular shape, the assembly of the two paddles when viewed from the top or bottom resembles a Y-shaped assembly. FIG. 8D illustrates an end view of the assembly of the first 510 and second 530 paddles along with the outer sleeve 550.

The outer sleeve 550 has substantially the same configuration as the outer sleeve 50 in the first embodiment. The outer sleeve 550 has slots 554a,b,c positioned to accommodate the shapes of the first 510 and second 530 paddles. As shown in FIG. 8C, the outer sleeve 550 slides over the assembly of the first 510 and second 530 paddles to complete the circumference of the desired shape corresponding to the shape of the access device to be inserted. The access device 200 (not shown) is then advanced over the outer sleeve 550. The entire assembly 500 including the first 510 and second 530 paddles and outer sleeve 550 may be removed simultaneously in one step leaving the access device 200 in the desired location.

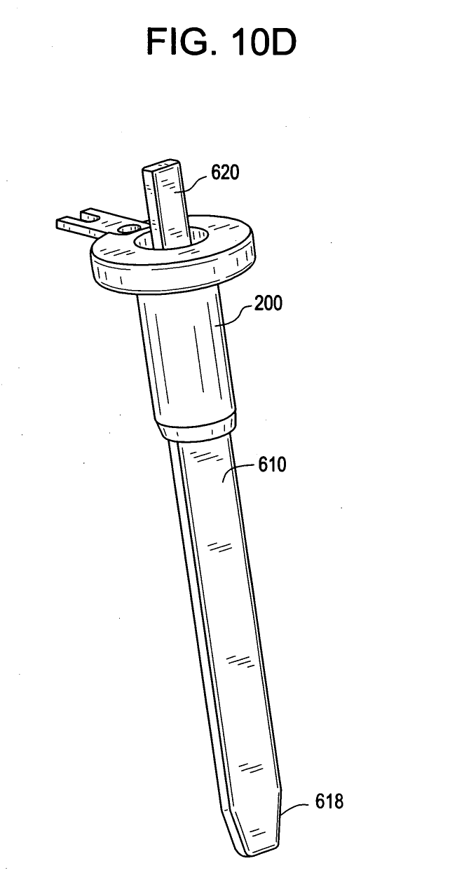

An alternate embodiment of the tissue expander system, illustrated in FIGS. 10A-D includes a first paddle 610. The paddle 610 extends from a proximal end 620 to a distal end 618 and has a generally rectangular shape with radiused edges. The distal end 618 may taper for ease of insertion. The paddle 610 has a width wₚ at its widest point and a width wₙₚ at its narrowest point. Shoulders 624a,b are formed where the paddle 610 changes width. The system also includes an outer sleeve 650 having a generally tubular shape with a central lumen 652 extending from a proximal end 660 to a distal end 658. The outer sleeve 650 has slots 654a,b extending proximally from the distal end 658. The central lumen 652 is sized to accommodate the narrow width portion 626 of the paddle 610 while the slots 654a,b are sized to receive the widest portion of the paddle 610. The length of each slot 654a,b is adapted to accommodate the shoulders 624a,b of the paddle 610. The outer diameter of the outer sleeve 650 may be the same as the inner diameter of the access device 200 to be used for the procedure.

The paddle 610 is inserted through a minimally invasive incision such that the width is parallel to the incision to the desired depth proximate a vertebral body. The paddle 610 is rotated approximately 90 degrees to stretch the tissue and muscle in all directions. The outer sleeve 650 may be inserted through the incision over the paddle 610 aligning the slots 654a,b with the shoulders 624a,b of the paddle 610. The outer sleeve 650 is advanced until it is proximate the vertebral body. The access device 200 may then be advanced over the assembly of the paddle 610 and the outer sleeve 650. FIG. 10C illustrates the assembly with the access device 200 in place. The paddle 610 and outer sleeve 650 may be removed simultaneously in one step by pulling on the proximal end 620 of the paddle. Alternately, the surgeon may choose to skip the outer sleeve and directly insert the access device 200 over the paddle 610 as shown in FIG. 10D.

While the tissue expander systems and methods of the present invention have been particularly shown and described with reference to the exemplary embodiments thereof, those of ordinary skill in the art will understand that various changes may be made in the form and details herein without departing from the spirit and scope of the present invention. Those of ordinary skill in the art will recognize or be able to ascertain many equivalents to the exemplary embodiments described specifically herein by using no more than routine experimentation. Such equivalents are intended to be encompassed by the scope of the present invention and the appended claims.

1. A tissue expander system comprising:
a first paddle extending from a proximal end to a distal end, the first paddle having a connecting feature; and
a second paddle having a connecting feature adapted to connect to the connecting feature of the first paddle forming an assembly of the first and second paddles.

2. The tissue expander system of claim 1 wherein the connecting feature of the first paddle is a channel.

3. The tissue expander system of claim 2 wherein the connecting feature of the second paddle frictionally fits within the channel of the first paddle.

4. The tissue expander system of claim 1 wherein the connecting features of the first and second paddles are angled with respect to each other.

5. The tissue expander system of claim 1 wherein the first and second paddles have an interlocking feature to connect the paddles.

6. The tissue expander system of claim 5 wherein the interlocking feature between the first and second paddle is a tongue and groove and wherein the channel of the first paddle has a tongue configuration and the second paddle has a complementary groove shape to engage the tongue.

7. The tissue expander system of claim 1 wherein the connecting feature of the first paddle is a shaped projection and the connecting feature of the second paddle is a complementary shaped channel.

8. The tissue expander system of claim 1 wherein the distal end of the first paddle further comprises a docking feature.

9. The tissue expander system of claim 8 wherein the docking feature is selected from the group consisting of serrated edges, teeth and roughened surface.

10. The tissue expander system of claim 1 wherein the first paddle and the second paddle have the same width.

11. The tissue expander system of claim 1 wherein the second paddle has a width greater than the first paddle.

12. The tissue expander system of claim 1 wherein the first paddle has a width greater than the second paddle.

13. The tissue expander system of claim 1 wherein the first paddle has a generally rectangular shape.

14. The tissue expander system of claim 1 wherein the first paddle has a generally v-shape.

15. The tissue expander system of claim 1 further comprising:
an outer sleeve adapted to slide over the first and second paddle assembly.

16. The tissue expander of claim 15, wherein the outer sleeve has at least one slot extending from a distal end of the outer sleeve adapted to receive the first paddle.

17. The tissue expander of claim 16, wherein the outer sleeve has a second slot extending from a distal end of the outer sleeve adapted to receive the second paddle.

18. The tissue expander system of claim 1 further comprising a handle.

19. A tissue expander system comprising:
a first paddle having a channel,
a second paddle having a channel adapted to mate with the channel of the first paddle to form an assembly, and
an outer sleeve having slots adapted to receive the first and second paddles.

20. The tissue expander system of claim 19 further comprising a modular handle.

21. A method of creating a minimally invasive pathway to a vertebral body comprising:
inserting a first paddle through an incision into proximity with the vertebral body;
inserting a second paddle through the incision;
connecting the second paddle to the first paddle;
inserting an outer sleeve over the first and second paddles into proximity with the vertebral body; and
placing an access device over the outer sleeve.

22. The method of claim 21 further comprising creating an incision for an anterior surgical approach to the vertebral body.

23. The method of claim 21 further comprising creating an incision for a posterior surgical approach to the vertebral body.

24. The method of claim 21 further comprising creating an incision for a lateral surgical approach to the vertebral body.

25. The method of claim 21 wherein the first paddle has a connecting feature and the second paddle has a connecting feature adapted to connect the first paddle to the second paddle.

26. The method of claim 21 further comprising:
attaching a handle to the first paddle.

27. The method of claim 21 further comprising:
rotating the first paddle within the incision.

28. The method of claim 21 further comprising:
attaching a handle to the second paddle.

29. The method of claim 21 further comprising:
simultaneously removing the first paddle, the second paddle and the outer sleeve.

30. The method of claim 21 further comprising:
inserting a guide wire to a desired site on the vertebral body; and
inserting the first paddle over the guide wire through the incision.

31. A method of creating a minimally invasive pathway to a vertebral body comprising:
inserting a paddle through an incision to a point proximate the vertebral body;
rotating the paddle within the incision;
inserting an access device over the paddle to create a pathway to the vertebral body.

32. The method of claim 31 further comprising:
inserting an outer sleeve over the paddle.

33. A method of creating a minimally invasive pathway to a vertebral body comprising:
inserting a paddle through an incision to a point proximate the vertebral body;
inserting a second paddle within the incision in connection with the first paddle;
and inserting an access device over the first and second paddles to create a pathway to the vertebral body.
